(19) United States
(12) Patent Application Publication
(10) Pub. No.: US 2004/0184963 A1
(43) Pub. Date: Sep. 23, 2004

(54) Recirculating hyperpolarized gas cells and associated methods of producing optically pumped hyperpolarized gas

(76) Inventor: Patrick A. Cella, Raleigh, NC (US)

Correspondence Address: Amersham Health, Inc, IP Department, 101 Carnegie Center, Princeton, NJ, 08540, US

(21) Appl. No.: 10/760,746

(22) Filed: Jan. 20, 2004

Related U.S. Application Data
(60) Provisional application No. 60/440,780, filed on Jan. 17, 2003.

(51) Int. Cl. B32B 5/02; G01N 7/00; B32B 27/12; G01N 33/00; B32B 27/04; G01N 27/00; G01N 21/00; A61N 1/30; A61B 5/05; F01N 3/10; G01N 31/00; G01N 24/00

(52) U.S. Cl. 422/83; 422/173; 422/174; 600/420; 604/20; 436/173

(57) ABSTRACT

Methods, systems, assemblies, computer program products and devices produce hyperpolarized gas by: (a) heating a target gas in an optical pumping cell, the optical pumping cell having opposing top and bottom portions; (b) polarizing the target gas in the optical pumping cell; (c) directing heated polarized gas to flow out of the top portion of the optical pumping cell to a storage reservoir, the storage reservoir having a temperature that is less than the temperature of the heated target gas flowing out of the optical pumping chamber; and (d) flowing previously polarized gas from the reservoir into the optical pumping cell.

### RELATED APPLICATIONS

This application claims the benefit of priority of U.S. Provisional Patent Application Serial No. 60/440,780, filed 17 Jan. 2003, the contents of which are hereby incorporated by reference as if recited in full herein.

### FIELD OF THE INVENTION

The present invention relates to the production of polarized noble gases that are particularly useful for NMR and magnetic resonance imaging (“MRI”) applications.

### BACKGROUND OF THE INVENTION

Polarized inert noble gases can produce improved MRI images of certain areas and regions of the body that have heretofore produced less than satisfactory images in this modality. Polarized helium-3 (“³He”) and xenon-129 (“¹²⁹Xe”) have been found to be particularly suited for this purpose. Unfortunately, as will be discussed further below, the polarized state of the gases is sensitive to handling and environmental conditions and can, undesirably, decay from the polarized state relatively quickly.

Hyperpolarizers are used to produce and accumulate polarized noble gases. Hyperpolarizes artificially enhance the polarization of certain noble gas nuclei (such as ¹²⁹Xe or ³He) over the natural or equilibrium levels, i.e., the Boltzmann polarization. Such an increase is desirable because it enhances and increases the MRI signal intensity, allowing physicians to obtain better images of the substance in the body. See U.S. Pat. Nos. 5,545,396; 5,642,625; 5,809,801; 6,079,213, and 6,295,834; the disclosures of these patents are hereby incorporated by reference herein as if recited in full herein.

In order to produce the hyperpolarized gas, the noble gas is typically blended with optically pumped alkali metal vapors such as rubidium (“Rb”). These optically pumped metal vapors collide with the nuclei of the noble gas and hyperpolarize the noble gas through a phenomenon known as “spin-exchange.” The “optical pumping” of the alkali metal vapor is produced by irradiating the alkali-metal vapor with circularly polarized light at the wavelength of the first principal resonance for the alkali metal (e.g., 795 nm for Rb). Generally stated, the ground state atoms become excited, then subsequently decay back to the ground state. Under a modest magnetic field (10 Gauss), the cycling of atoms between the ground and excited states can yield nearly 100% polarization of the atoms in a few microseconds. This polarization is generally carried by the lone valence electron characteristics of the alkali metal. In the presence of non-zero nuclear spin noble gases, the alkali-metal vapor atoms can collide with the noble gas atoms in a manner in which the polarization of the valence electrons is transferred to the noble-gas nuclei through a mutual spin flip “spin-exchange.”

Generally stated, as noted above, conventional hyperpolarizers include an optical pumping chamber held in an oven and in communication with a laser source that is configured and oriented to transmit circularly polarized light into the optical pumping chamber during operation. The hyperpolarizers may also monitor the polarization level achieved at the polarization transfer process point, i.e., at the optical cell or optical pumping chamber. In order to do so, typically a small “surface” NMR coil can be positioned adjacent the optical pumping chamber to excite and detect the gas therein and thus monitor the level of polarization of the gas during the polarization-transfer process. See U.S. Pat. No. 6,295,834 for further description of polarization monitoring systems for optical pumping cells and polarizers.

On-board hyperpolarizer monitoring equipment no longer requires high-field NMR equipment, but instead can use low-field detection techniques to perform polarization monitoring for the optical cell at much lower field strengths (e.g., 1-100G) than conventional high-field NMR techniques. This lower field strength allows correspondingly lower detection equipment operating frequencies, such as 1-400kHz. Saam et al. has proposed a low-frequency NMR circuit expressly for the on-board detection of polarization levels for hyperpolarized ³He at the optical chamber or cell inside the temperature-regulated oven that encloses the cell. See Saam et al., Low Frequency NMR Polarimeter for Hyperpolarized Gases, Jnl. of Magnetic Resonance 134, 67-71 (1998), the contents of which are hereby incorporated by reference as if recited in full herein.

Polarizing the target gas using spin exchange optical pumping is a relatively slow process; it can take about 10-16 hours, or longer, for a 1-liter batch of polarized helium gas to reach or approach its saturation polarization in conventionally sized polarization cells. Using larger volume cells can require larger ovens, more laser power, and stronger optics than those conventionally used.

Thus, there remains a need for methods and systems that can provide increased volume production of polarized gas.

### SUMMARY OF THE INVENTION

In view of the foregoing, embodiments of the present invention provide hyperpolarizers, systems, methods, and computer program products to provide increased amounts of polarized gas.

Certain embodiments are directed to methods for producing hyperpolarized gas by: (a) heating a target gas in an optical pumping cell, the optical pumping cell having opposing top and bottom portions; (b) polarizing the target gas in the optical pumping cell; (c) directing heated polarized gas to flow out of the top portion of the optical pumping cell to a storage reservoir, the storage reservoir having a temperature that is less than the temperature of the heated target gas flowing out of the optical pumping chamber; and (d) flowing previously polarized gas from the reservoir into the optical pumping cell.

Other embodiments are directed to polarizing systems for polarizing a target gas via spin-exchange with an optically pumped alkali metal. The systems include: (a) an optical pumping cell having separate exit and inlet ports, the exit port residing on a top portion of the optical pumping cell; (b) a reservoir chamber in fluid-communication with the optical pumping cell, the reservoir having separate exit and inlet ports; and (c) a circulation gas flow path extending from the optical pumping cell exit port to the reservoir inlet port and the reservoir exit port to the optical pumping cell inlet port, wherein, in operation, target gas is polarized in the optical pumping cell and the polarized target gas is convectively flowed out of the optical pumping cell inlet port into the gas flow path to the reservoir chamber.

Still other embodiments are directed to apparatus for polarizing a target gas, including: (a) means for heating a target gas in an optical pumping cell, the optical pumping cell having opposing top and bottom portions; (b) means for polarizing the target gas in the optical pumping cell; (c) means for convectively flowing heated polarized gas out of the top portion of the optical pumping cell to a storage reservoir, the storage reservoir having a temperature that is less than the temperature of the heated target gas flowing out of the optical pumping chamber; and (d) means for flowing previously polarized gas from the reservoir into the optical pumping cell.

Still other embodiments are directed to computer program products for operating a hyperpolarizer with a laser excitation source. The hyperpolarizer employs convection-induced flow discharge of polarized gas from at least one optical pumping cell to at least one reservoir using a circulating gas flow path that includes a dispensing valve and a flow rate valve in fluid communication with the circulating gas flow path to produce polarized noble gas. The computer program product comprises computer readable storage medium having computer readable program code embodied in the medium. The computer-readable program code including: (a) computer readable program code that determines the polarization level of polarized gas held in the optical pumping cell of the hyperpolarizer; (b) computer readable program code that determines the polarization level of polarized gas held in the reservoir of the hyperpolarizer; and (c) computer readable program code that directs the optical pumping cell to optically pump target gas and previously polarized target gas.

Advantageously, the present invention can provide increased timely production of hyperpolarized gas where reservoirs can hold individual patient-sized quantities (such as 0.5-2 liters) of polarized gas while the optical pumping cell is operating to produce a fresh supply of polarized gas. Unlike batch type production processes, during operation, the systems and methods provided by the instant invention can substantially continuously recirculate polarized gas and/or dispense and supply fresh target gas as desired to provide increased amounts of polarized gas using a polarization cell that is sized similar to conventional cells.

All or selected operations, functions and/or configurations of the embodiments described above with may be carried out as methods, systems, computer program products, assemblies and/or devices as contemplated by the present invention.

The foregoing and other objects and aspects of the present invention are explained in detail herein.

### BRIEF DESCRIPTION OF THE DRAWINGS

FIG. 1 is a block diagram of operations that can be used to carry out embodiments of the present invention.

FIG. 2 is a schematic illustration of a polarizer system having an optical pumping cell with a closed loop flow path for circulating polarized gas according to embodiments of the present invention.

FIG. 3 is a schematic illustration of a polarizer system similar to that shown in FIG. 2, but with the reservoir located below the optical cell, according to embodiments of the present invention.

FIG. 4 is a schematic illustration of a polarizer system according to other embodiments of the present invention.

FIG. 5 is a block diagram of a computer module for use with convectively discharged gas according to embodiments of the present invention.

FIG. 6 is a front partially cutaway view of a solenoid configured to provide a magnetic field according to embodiments of the present invention.

### DETAILED DESCRIPTION OF EMBODIMENT OF THE INVENTION

The present invention will now be described more fully hereinafter with reference to the accompanying figures, in which preferred embodiments of the invention are shown. This invention may, however, be embodied in many different forms and should not be construed as limited to the embodiments set forth herein. Like numbers refer to like elements throughout. In the drawings, layers, regions, or components may be exaggerated for clarity. In the figures, broken lines indicate optional features unless described otherwise.

In the description of the present invention that follows, certain terms may be employed to refer to the positional relationship of certain structures relative to other structures. As used herein the term “forward” and derivatives thereof refer to the general direction the target gas or target gas mixture travels as it moves through the hyperpolarizer system; this term is meant to be synonymous with the term “downstream,” which is often used in manufacturing environments to indicate that certain material being acted upon is farther along in the manufacturing process than other material. Conversely, the terms “rearward” and “upstream” and derivatives thereof refer to the directions opposite, respectively, the forward and downstream directions.

Also, as described herein, polarized gases are produced and collected and may, in particular embodiments, be frozen, thawed, be used alone and/or combined with other constituents, for MRI and/or NMR spectroscopy applications. For ease of description, the term “frozen polarized gas” means that the polarized gas has been frozen into a solid state. The term “liquid polarized gas” means that the polarized gas has been or is being liquefied into a liquid state. Thus, although each term includes the word “gas,” this word is used to name and descriptively track the gas that is produced via a hyperpolarizer to obtain a polarized “gas” product. Thus, as used herein, the terms “gas” or “target gas” may be used in certain places to descriptively indicate a hyperpolarized noble gas product and may be used with modifiers such as “solid”, “frozen”, and “liquid” to describe the state or phase of that product. As also used herein, the terms “polarized gas”, “target gas” and/or “polarized target gas” include at least one intended target gas of interest (such as, but not limited to, ³He and/or ¹²⁹Xe) and may include one or more other constituents such as other carrier or blending gases, buffer gases, or carrier liquids as desired. Further, the terms “polarize”, “polarizer”, “polarized”, and the like are used interchangeably with the terms “hyperpolarize”, “hyperpolarizer”, “hyperpolarized” and the like.

Various techniques have been employed to accumulate and capture polarized gases. For example, U.S. Pat. No. 5,642,625 to Cates et al. describes a high volume hyperpolarizer for spin -exchange polarized noble gas and U.S. Pat. No. 5,809,801 to Cates et al. describes a cryogenic accumulator for spin-polarized ¹²⁹Xe. As used herein, the terms “hyperpolarize,” “polarize,” and the like, are used interchangeably and mean to artificially enhance the polarization of certain noble gas nuclei over the natural or equilibrium levels. Such an increase is desirable because it allows stronger imaging signals corresponding to better MRI images of the substance and a targeted area of the body. As is known by those of skill in the art, hyperpolarization can be induced by spin-exchange with an optically pumped alkali-metal vapor or alternatively by metastability exchange. See Albert et al., U.S. Pat. No. 5,545,396.

Generally described, hyperpolarizer systems include optic systems with a laser source, such as a diode laser array, and optic beam forming or focusing components, such as beam splitters, lenses, mirrors or reflectors, beam or wave polarizers or wave shifters, and/or other focal components for providing the circularly polarized light source to the target gas held in an optical pumping cell. For ease of description, the term “optic system” as used herein includes the optical pumping components used to generate and/or focus the circularly polarized light.

Generally described, operations of the present invention are carried out using temperature differentials to provide a convection-induced discharge of polarized gas from the optical pumping cell to thereby cause the polarized gas to rise out of the optical pumping cell and flow to a reservoir. In certain embodiments, the systems include a closed loop circulating gas flow path that connects the optical pumping cell and the reservoir and allows gas in the reservoir to travel back to the optical pumping cell to be re-polarized as needed.

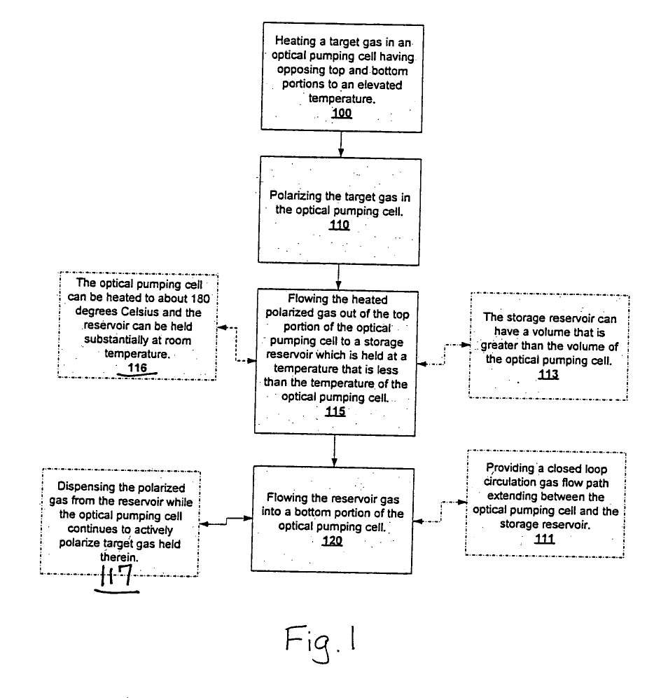

FIG. 1 illustrates operations that can be used to carry out embodiments of the present invention. A target gas can be heated in an optical pumping cell that has opposing top and bottom portions (block 100). The target gas can be polarized in the optical pumping cell (block 110). The heating can be carried out using any suitable energy source or sources, such as by using one or more of: (a) the laser energy used to optically pump the target gas; (b) a strip or plate heater; (c) an oven encasing the optical pumping cell; or (d) another desired heat-inducing source. The optical pumping cell (and gas therein) can be heated to at least about 150 degrees Celsius, and typically to about 180 degrees Celsius (block 116), and the reservoir can be at approximately room temperature.

In any event, the heated polarized gas flows out of the top portion of the optical pumping cell to a storage reservoir which is held at a temperature that is less (below) that of the optical pumping cell (block 115). The reservoir gas is flowable back into the optical pumping cell (block 120) at a location that is different from where the gas flows out of the pumping cell. Typically, the reservoir gas is directed to flow into a bottom portion of the pumping cell, although other locations may also be used. However, the location of the inlet should be spaced apart a vertical distance from the outlet sufficient to allow only the more recently polarized (heated) gas to exit the outlet port.

The storage reservoir can be sized and configured with a volume that is at least as large as the optical pumping cell, and typically the reservoir volume will be about the same or greater than that of the optical pumping cell (block 113). In certain particular embodiments, the optical pumping cell may have an internal volume of about 250 cc's and the reservoir can have an internal volume of between about 250-1000cc's.

In certain embodiments, a closed loop gas circulating flow path can be configured to extend between the optical pumping cell and the reservoir (block 111). The polarized gas can be dispensed from the reservoir or at another location in fluid communication with the circulation gas flow path downstream of the reservoir and upstream of the optical pumping cell. The dispensing can be carried out while the optical pumping cell is actively operating to polarize target gas held therein (block 117). As the reservoir is held at ambient temperature, there is no need to cool the polarized gas before dispensing from the reservoir or downstream thereof as noted above. In operation, the circulating gas flow path, the optical cell and the reservoir can all be maintained at a common system pressure, typically between about 6-10 atm, and more typically about 8-10 atm. However, pressures of up to between about 20-30 atm may be used with suitable hardware. During set-up, the system can be charged to be at the desired system pressure by adding suitable amounts of target and supplemental gases.

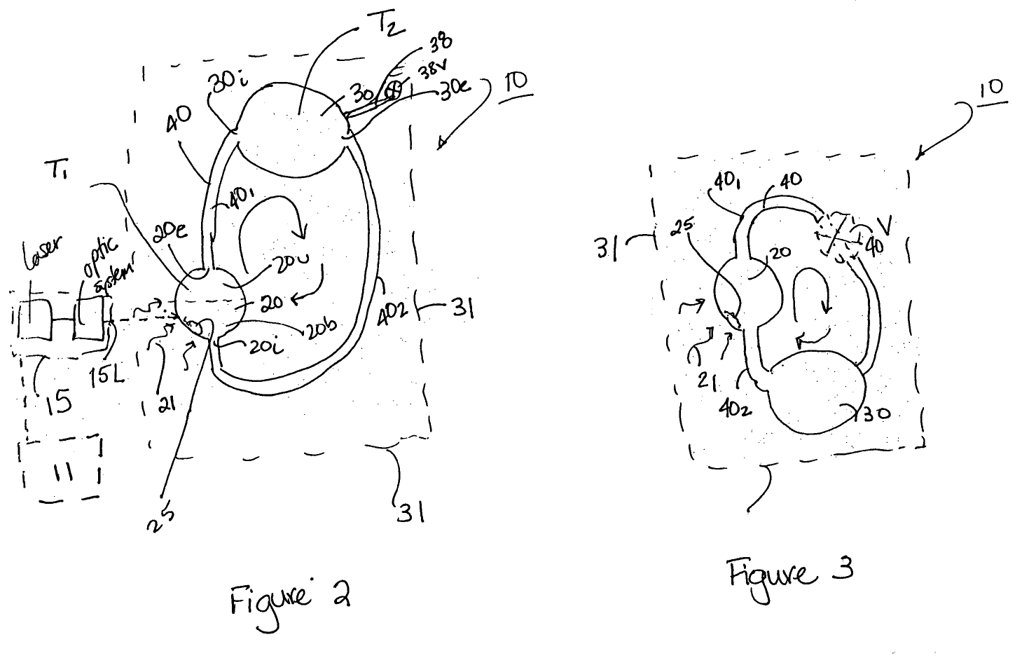

Turning now to FIG. 2, one example of a hyperpolarizer system 10 is shown. In this embodiment, the system 10 includes an optic system 15 that generates and transmits polarized light 15L, an optical pumping cell 20, a reservoir 30, a circulating gas flow path 40 and a magnetic field 31 generated by a magnetic field source. The magnetic field 31 can be configured with a low field strength having a field size and sufficient homogeneity to extend to cover both the optical pumping cell 20 and the holding cells 30. In operation, the gas flows in the direction illustrated by the arrow drawn in the center of the figure. The flow direction can be changed by altering the configuration of the reservoir 30 with respect to the optical pumping cell 20.

The optical pumping cell 20 includes opposing top and bottom portions 20u, 20b, located on opposite sides of a line drawn through the center of the cell 20. A small quantity of alkali metal 25, such as rubidium, can be positioned in the cell 20 for conversion into a vapor during spin-exchange. The cell 20 includes an exit port 20e and a spaced apart inlet port 20i. The exit port 20e should be positioned on the upper portion 20u of the cell 20 to take advantage of the convection-induced discharge of the heated polarized gas from the cell 20. That is, as hot polarized gas rises, it will flow out of the exit port 20e and into the gas flow path 40 as it advances to the reservoir 30 (at a reduced temperature relative to the optical pumping cell 20). The exit port 20e should be positioned above the inlet 20i on the cell body and each port can operate at the same pressure. The optical pumping cell 20 may be sized with an internal volume of between about 100-500 cc's, and is typically about 250 cc's. In certain embodiments, the optical pumping cell 20 and reservoir 30 can be shaped to be substantially spherical to increase the ratio of surface area to volume of the container. However, other shapes may be used where appropriate.

The reservoir 30 includes a gas flow inlet 30i and a spaced apart outlet 30e. The body of the reservoir 30 may be sized to be about the same size as the cell 20. More typically, the reservoir 30 is configured with a volume that is increased relative to the optical pumping cell 20 so that it is able to hold several successive polarized batches of gas therein while releasing polarized gas from the exit port 30e (or a dispensing port 38. For example, for a system 10 that includes an optical pumping cell 20 having a volume of between about 100-500 cc's, the reservoir can be sized to be about 1.5-4 times the size of the cell 20, and typically 2-3 times the size of the cell 20, for example, the reservoir 30 can be between about 300-1000 cc's, or greater.

The reservoir 30 may be cooled or be at ambient temperature. In any event, the reservoir 30 is held at a temperature (T₂) that is less than that of the optical pumping cell 20 and/or gas therein (T₁) to provide a desired thermal gradient in the closed loop system. As noted above, the heat 21 to the optical pumping cell can be supplied by any suitable source to provide the desired T₁ and/or thermal gradient.

The source should be configured to inhibit the decay of polarized gas. As non-limiting examples, the source may be blown hot air, a surface wrap heater, laser energy (from the polarizing instrumentation or supplemental energy source), and the like. An insulating oven (not shown) may be positioned over the optical pumping cell 20. For example, the optical cell 20 can be housed in a re-circulating oven configuration that holds a heating element in a remote location and blows forced hot air to the cell 20 to inhibit any depolarizing influences from proximity of the gas (not shown). In other embodiments, the laser energy is captured in a thermally insulated oven space, providing a substantially self-heating configuration that harnesses the heat released by the optical pumping process.

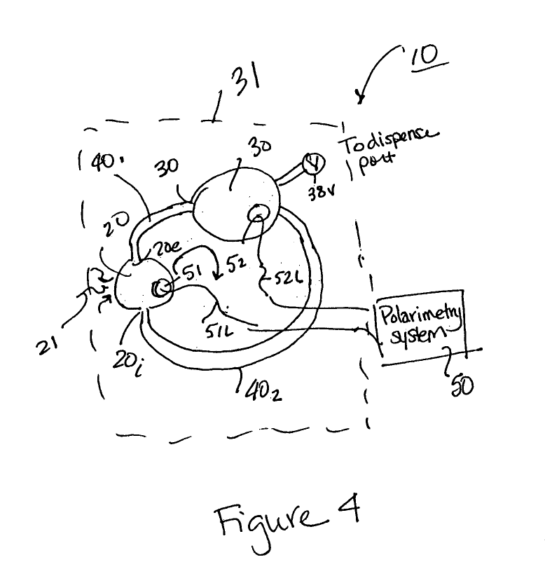

The heated polarized gas exits the top portion of the optical pumping cell 20 and travels in the gas flow path 40 to the reservoir 30. The reservoir 30 may be located above the cell 20 (FIG. 2) or below the reservoir 30 (FIG. 3). The reservoir 30 may also be located at the same height with the gas flow segment 40₁ rising out of the optical pumping cell 20 to connect it to the reservoir 30 (not shown).

The gas flow path 40 may be configured from a small diameter tubing or conduit configured to withstand the desired system pressures. The small diameter tubing may have about a 0.25-inch diameter or less. The gas flow path 40 can be sized to provide a desired flow rate of the gas traveling in the closed loop circulation path 40. The gas flow path 40 can be formed from any suitable polarization decay-inhibiting material such as an aluminosilicate or sol-gel coated glass tube. Other suitable materials may also be used, such as, but not limited to, anodized aluminum.

The gas flow path 40 may be sized with a first flow segment 40₁ that has a smaller length and/or a reduced volume between the optical pumping cell 20 and the reservoir 30 (the outbound leg) relative to a second flow segment 40₂, extending between the reservoir 30 and the optical pumping cell 20 (the return leg). This configuration may reduce the amount of polarization decay that the polarized gas experiences as it travels to the reservoir 30. In other embodiments, the legs 40₁, 40₂ can be substantially the same length, or, as shown in FIG. 3, the second (return) leg 402 can be shorter.

In certain embodiments, the inlet 30i of the reservoir 30 is spaced apart at least about 2 inches from the exit port 20e of the optical pumping cell 20. In particular embodiments, the first leg of the gas flow path 40₁ can be about 2-3 inches long. In addition, the second leg of the gas flow path 40₂ may be about 3-6 times the length of the first leg 40₁. For example, the second leg 40₂ can be about 14 inches long.

The associated gas flow path 40 (comprising legs 40₁ and 40₂) in the system 10 may have a volume that is substantially less than that of the combined volumes of the reservoir 30 and cell 20. Substantially less means that the flow path may have a volume that is about 10% the total flow path volume of the system. The total flow path volume includes the volume of the legs 40₁, 40₂ and the volume of the reservoir 30 and cell 20. For example, the flow path volume 40 when measured to exclude the cell 20 and reservoir 30 can be about 15 cc's when the flow path 40 with the cell 20 and reservoir can have a volume of between about 500-1250 cc's.

The cross-sectional width of the first leg 40₁ may be sized to be different than the second leg 40₂ for flow rate purposes. In other embodiments, the first and second legs 40₁, 40₂ may be configured to be substantially the same cross-sectional width.

FIG. 3 also illustrates that the gas flow path 40 may include an optional flow control valve 40V. The valve 40V can be positioned in advance of the reservoir, after the reservoir 30 in advance of the optical pumping cell 20. In lieu of one valve, a plurality of valves can also be used along the gas flow path 40 (not shown). Polarization friendly valves should be used, particularly upstream of the reservoir 30 and downstream of the cell 20, where maintenance of the polarization strength is desired.

In certain embodiments, the circulating gas flow path 40 can be configured to provide a flow rate of about 50 cc's/min from the optical cell 20 to the reservoir 30 and/or from the reservoir 30 to the optical cell 20. In particular embodiments, the entire closed loop path 40 (the closed loop gas flow path includes the cell 20 and the reservoir 30) are held at the same pressure and the gas travels in the flow path 40 with a substantially constant flow rate.

FIG. 2 illustrates that a dispensing port 38 and associated dispensing valve 38V may be positioned on the reservoir 30 to allow polarized gas to be dispensed into a desired delivery container. The dispensing port 38 may also be located downstream or upstream of the reservoir 30 in fluid communication with the gas flow path 40. Placing the dispensing port at the reservoir 30 (or downstream but in proximity thereto) can allow the polarized gas to be cooled prior to dispensing (no longer requiring the polarization cell 20 to be cooled after batch production of ³He). Because the optical pumping cell does not have to be cooled between batches of polarized gas, the optical pumping cell can produce additional amounts of polarized gas, thereby obviating the “down-time” associated with cool down therein.

After dispensing, additional target gas or a target gas blend can be added to the gas flow path 40 to replace the dispensed polarized gas. As such, a filling port (not shown) may be connected to the gas flow path 40 used to controllably re-supply the closed loop system. Thus, as noted before, the gas flow path 40 can be operably associated with one or more (automated) valves (identified with the letter “V”) in the travel path to allow for refill, dispensing, and/or to control the flow rate, pressure and the like of the gas in the closed loop system. In other embodiments, gas can continue to circulate at lower pressures and flow rates until substantially all of the polarized gas is dispensed. Then, the entire system 10 can be refilled.

As shown in FIG. 2, the polarization and/or flow of the gas can be automated and controlled by a controller 11. The controller 11 may also include computer program code with instructions that control the sequencing of operations and/or the activation of the optic system 15. The hyperpolarizer 10 may also include a gas flow path 40 and an associated dispense port 40p that can allow the polarized gas 50p to be dispensed. The controller 11 may be configured to automatically monitor the polarization level of the polarized gas in one or both of the optical pumping cell 20 and the reservoir 30. Further, the controller 11 may prohibit dispensing polarized gas that has decayed below a desired polarization level proximate in time to a planned and/or requested dispensing output. The controller 11 can monitor the temperature at certain locations in the closed loop circulation flow path and direct the adjustment (increase or decrease) of the temperature of either or both of the optical pumping cell or the reservoir to provide the desired thermal gradient and/or to increase or decrease the thermal gradient to increase or decrease the flow rate in the closed loop path 40.

FIG. 4 illustrates that the system 10 can include NMR coils 51, 52 and associated leads, 51L, 52L, respectively extending from the coils to a polarimetry system 50. It is noted that polarimetry systems are well known to those of skill in the art. The polarization strength of the polarized gas can be monitored using polarimetry and the RF polarimetry coil(s) 51, 52. See, e.g., U.S. Pat. No. 6,295,834 and U.S. patent application Ser. No. 09/334,341, and Saam et al., Low Frequency NMR Polarimeter for Hyperpolarized Gases, Jnl. of Magnetic Resonance 134, 67-71 (1998), the contents of each of which are hereby incorporated by reference as if recited in full herein.

The magnetic field 31 shown by the broken lines in FIGS. 2-4, which covers the optical pumping cell 20 and the reservoir 30, can be provided by any suitable magnetic field source, such as permanent magnets or electromagnets. A low magnetic field strength can be used, typically about 500 Gauss or less, and more typically about 100 Gauss or less, with sufficient homogeneity to inhibit depolarizing influences during production and storage of the polarized target gas. In particular embodiments, a field strength of between 7-20 Gauss may be appropriate. In certain embodiments, a magnetic field homogeneity on the order of 10^{31 3} cm^{31 1} (Gauss) is desirable, at least for the regions covering hyperpolarized gas for any length of time. Conventionally, Helmholtz coils have been used. The magnetic field 31 can also be configured to extend a distance sufficient to cover the gas dispensing port 38 (FIG. 2). In particular embodiments, the magnetic field 31 may be further generated, formed or shaped to extend to cover the receiving containers of polarized gas during dispensing (not shown).

Thus, the field source may be a pair of Helmholtz coils, as is well known to those of skill in the art and/or permanent magnets. In certain embodiments, as shown in FIG. 6, the field source is a cylindrical solenoid 80 that is configured to generate the magnetic field 31. The solenoid 80 can include a cavity 80c that is sized and configured to surround the optical pumping cell 20 and reservoir 30. The polarized gas can be dispensed by directing the gas to flow or dispense out the dispensing port 38 from the hyperpolarizer substantially along the axis of the solenoid 80. Other dispensing configurations can also be used. The solenoid 80 may be configured with 648 full winding layers and 58 extra layers of windings (16-guage wire) on each end portion (for a total of 1528 windings) providing an ellipsoid shaped magnetic field of about 8 inches wide and 18 inches long. The solenoid can be configured with about 3440 feet of wire (about 14.4 Ohms). Using 1.0 amp and 15V, a field of about 18.7 Gauss with sufficient size and homogeneity can be generated. Suitable solenoid field sources, such as end-compensated solenoid configurations, are described in co-assigned, co-pending U.S. patent application Ser. No. 09/333,571, and permanent magnet configurations are described in U.S. patent application Ser. No. 09/583,663; the contents of these applications are hereby incorporated by reference herein as if recited in full herein.

In certain embodiments, the reservoir 30, optical pumping cell 20, and flow path 40 therebetween, are all held within a region of sufficient homogeneity within a single common magnetic holding field B_{H} inside the cavity of the solenoid as shown in FIG. 6. In other embodiments, a plurality of separate magnetic field sources or generators (all electromagnets, all permanent magnets, or combinations of each) can be used, to provide the desired holding fields for the hyperpolarizer (not shown).

The cell 20 can be configured for producing the same type of hyperpolarized target gas, typically a noble gas, such as, but not limited to ³He or ¹²⁹Xe.

Before or during initial start-up, the closed loop system 10 can be filled or charged with target gas (typically a gas mixture) so that the cell 20 is above atmospheric pressures, typically at about 110 psi at room temperature. In operation, the cell can operate at elevated pressures such as between about 6-10 atm as noted above. In certain particular embodiments, instead of pre-filling the cell 20 and engaging it wit the system 10, the cell 20 can be filled with the desired target gas by directing a supply of exogenously held gas into the cell 20, such as by using the dispensing path and/or port or a fill port and path (not shown). See co-pending, co-assigned U.S. patent application Ser. Nos. 09/949,394; 10/277,911; 10/277,909; and U.S. Provisional Application Serial No. 60/398,033 (describing manifolds and filling and dispensing systems, as well as purge and evacuate procedures), the contents of which are hereby incorporated by reference as if recited in full herein.

To recharge the closed loop system after dispensing all or portions of a batch or batches of polarized gas, the system 10 can be configured to allow exogenous (non-polarized gas) refills, such as by flowing target gas into the gas flow path 40, the reservoir, or the cell 20. Typically, the refill gas will be directed to enter the system 10 downstream of the reservoir so as not to dilute the polarized gas held therein.

Generally described, in operation, the optical pumping cell 20 is heated to an elevated temperature, generally to about 150-200° C. or greater, and typically to about 180° C. The target gas mixture is polarized in the cell 20 at a pressure of between about 6-10 atm. Of course, as is known to those of skill in the art, with hardware capable of operating at increased pressures, operating pressures of above 10 atm, such as about 20-30 atm, can be used to pressure-broaden the alkali metal absorption and promote spin exchange. Using increased pressures with an alkali metal (such as rubidium (“Rb”)) can facilitate the absorption of the optical light (approaching up to 100%). In contrast, for laser line widths less than conventional line widths, lower pressures can be employed.

The optical pumping cell 20 typically includes a quantity of alkali metal 25 (FIG. 2) that vaporizes and cooperates to provide the spin-exchange polarization of the target gas of interest. The alkali metal can typically be used for a plurality of pumping procedures without replenishment. The optical pumping cell 20 has conventionally been formed from a substantially pure (substantially free of paramagnetic contaminants) aluminosilicate glass because of its ability to withstand deterioration due to the corrosive potential of alkali metal and its relatively friendly treatment of the hyperpolarized state of the gas (i.e., “good spin relaxation properties”—so stated because of its ability to inhibit surface contact-induced relaxation attributed to collisions of the gas with the walls of the cell). Coatings such as sol-gel coatings, deuterated polymer coatings, metal film coatings and other coatings and materials that inhibit depolarization have also been proposed. See, e.g., U.S. patent application Ser. No. 09/485,476 and U.S. Pat. No. 5,612,103, the contents of each of which are hereby incorporated by reference as if recited in full herein. The reservoir 30 may be formed of similar materials that inhibit polarization decay.

During polarization, the noble gas of choice (such as ³He) is held in the optical cell along with the alkali metal. The optical pumping cell is exposed to elevated pressures and heated in an oven to a high temperature as a light source, typically provided by a laser and/or laser array in an optic system 15 (FIG. 2), is directed into the optical cell 20 to optically pump the alkali metal and polarize the target gas.

The system 10 may employ helium buffer gas in the optical pumping cell 20 to pressure broaden the Rb vapor absorption bandwidth. The selection of a buffer gas can be important because the buffer gas—while broadening the absorption bandwidth—can also undesirably impact the alkali metal-noble gas spin-exchange by potentially introducing an angular momentum loss of the alkali metal to the buffer gas rather than to the noble gas as desired.

As will be appreciated by those of skill in the art, Rb is reactive with H₂O. Therefore, any water or water vapor introduced into the polarizer cell 20 can cause the Rb to lose laser absorption and decrease the amount or efficiency of the spin-exchange in the polarizer cell 20. Thus, as an additional precaution, an extra filter or purifier (not shown) can be positioned before the inlet of the polarizer cell 20 with extra surface area to remove even additional amounts of this undesirable impurity in order to further increase the efficiency of the polarizer.

The hyperpolarizer system 10 can also capitalize on the temperature change in the line between the heated pumping cell 20 and the reservoir 30 or in the reservoir 30 itself to precipitate the alkali metal 25 from the polarized gas stream in the cell 20 and/or in the conduit proximate the cell 20 that forms a part of the gas flow path.

As will be appreciated by one of skill in the art, the alkali metal 25 can precipitate out of the gas stream at temperatures of about 40° C. The system 10 can also include an alkali metal reflux condenser (not shown) or post-cell filter (not shown). The refluxing condenser can employ a vertical refluxing outlet pipe, which is kept at room temperature. The gas flow velocity through the refluxing pipe and the size of the refluxing outlet pipe is such that the alkali metal vapor condenses and drips back into the pumping cell by gravitational force. Alternatively, and/or in addition, an Rb filter can be used to remove excess Rb from the hyperpolarized gas prior to collection or accumulation along the dispensing path or at the dispensing port 38 (FIG. 2). In any event, it is desirable to remove alkali metal prior to delivering (and, typically, prior to dispensing from the hyperpolarizer) the polarized gas to a patient to provide a non-toxic, sterile, or pharmaceutically acceptable substance (i.e., one that is suitable As will be understood by those of skill in the art, in certain embodiments, the controller 11 can be configured to provide the purge/pump capacity from a central purge gas source and vacuum pump to the closed loop gas flow path 40 to clean the system of contaminants. As such, fluid flow paths of plumbing extending between the purge and vacuum sources to the optical pumping cell(s) 20 and/or reservoir 30 can be defined by a fluid distribution system or manifold network of plumbing, valves, and solenoids. These fluid flow paths selectively direct purge gas to and from the optical pumping cell 20 reservoir 30, and closed loop gas flow path 40 to purge and evacuate the flow paths in order to prepare them for polarization operations or to hold or process polarized gas.

The quantity of target gas can be sized so as to provide the constituents commensurate with that needed to form a single batch. Typically, the unpolarized target gas is a gas mixture that comprises a minor amount of the target noble gas and a larger quantity of one or more high purity biocompatible filler gases. For example, for ³He polarization, an unpolarized gas blend of ³He/N₂ can be about 99.25/0.75. For producing hyperpolarized ¹²⁹Xe, the pre-mixed unpolarized gas mixture can be about 85-98% He (preferably about 85-89% He), about 5% or less ¹²⁹Xe, and about 1-10% N₂ (preferably about 6-10%).

Discrete amounts of polarized gas can be meted out of the reservoir 30 in quantities that provide a single patient amount for a single MRI imaging or NMR evaluation session. To provide the pharmaceutical grade polarized gas doses, the polarized gas itself may be mixed with pharmaceutical grade carrier gases or liquids upon dispensing, or may be configured to be administered as the only or primary substance or constituent. In particular embodiments, the polarized gas is ³He and is mixed with nitrogen filler gas prior to or during dispensing (or before administration to a patient) to form a volume of gas blend to be inhaled by the patient. In other embodiments, for example, for producing inhalable ¹²⁹Xe, the 1²⁹Xe may form a major portion (or all) of the administered dose. In other embodiments, the polarized gas can be formulated to be injected in vivo (in a liquid carrier, in microbubble solution, or in gaseous form).

The hyperpolarizer system 10 can include one or more purifiers or filters (not shown) that are positioned in line with the plumbing to remove impurities such as water vapor, alkali metal, and oxygen from the system (or to inhibit their entry therein). The hyperpolarizer system 10 can also include various sensors including, a flow meter, as well as a plurality of valves, electrical solenoids, hydraulic, or pneumatic actuators that can be controlled by the controller 11 to define the fluid flow path and operation of the components of the hyperpolarizer 10. As will be understood by those of skill in the art, other flow control mechanisms and devices (analog and electronic) may be used within the scope of the present invention. For additional descriptions of meted dispensing systems, see co-pending U.S. patent application Ser. Nos. 10/277,911 and 10/277,909 and U.S. Provisional Application Serial No. 60/398,033, the contents of which are hereby incorporated by reference as if recited in full herein.

The hyperpolarizer system 10 can be located at the point of use site (hospital or clinic) typically in the vicinity of or proximate to the MRI or NMR equipment. That is, the hyperpolarizer system 10 can reside adjacent the MRI suite or in a room of a wing proximate thereto so as to limit the spatial transport and potential exposure to undesirable environmental conditions. In certain embodiments, the polarized gas transport time between the hyperpolarizer and the imaging suite is less than about 1 hour. Placing the hyperpolarizer in the clinic or hospital allows for short and consistent transport times procedure to procedure. In addition, formulating the pharmaceutical polarized gas with a polarized gas having higher levels of polarization can reduce the amount of the polarized gas used to form the end dose product, thereby potentially reducing the cost of the product.

As will be appreciated by one of skill in the art, the present invention may be embodied as a method, data or signal processing system, or computer program product. Accordingly, the present invention may take the form of an entirely hardware embodiment, an entirely software embodiment or an embodiment combining software and hardware aspects. Furthermore, the present invention may take the form of a computer program product on a computer-usable storage medium having computer-usable program code means embodied in the medium. Any suitable computer readable medium may be utilized including hard disks, CD-ROMs, optical storage devices, or magnetic storage devices.

The computer-usable or computer-readable medium may be, for example but not limited to, an electronic, magnetic, optical, electromagnetic, infrared, or semiconductor system, apparatus, device, or propagation medium. More specific examples (a nonexhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted or otherwise processed in a suitable manner if necessary, and then stored in a computer memory.

Computer program code for carrying out operations of the present invention may be written in an object oriented programming language such as Java7, Smalltalk, Python, or C++. However, the computer program code for carrying out operations of the present invention may also be written in conventional procedural programming languages, such as the “C” programming language or even assembly language. The program code may execute entirely on the user's computer, partly on the user's computer, as a stand-alone software package, partly on the user=s computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider).

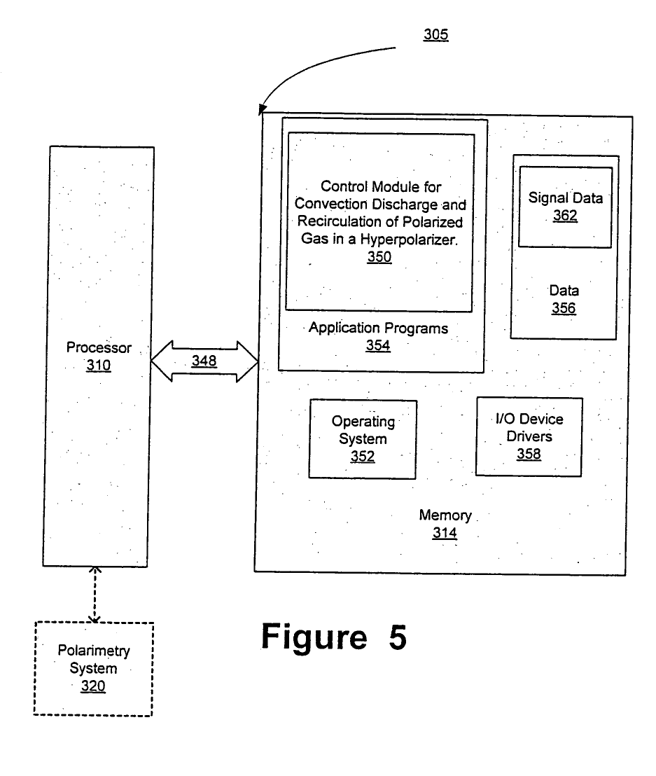

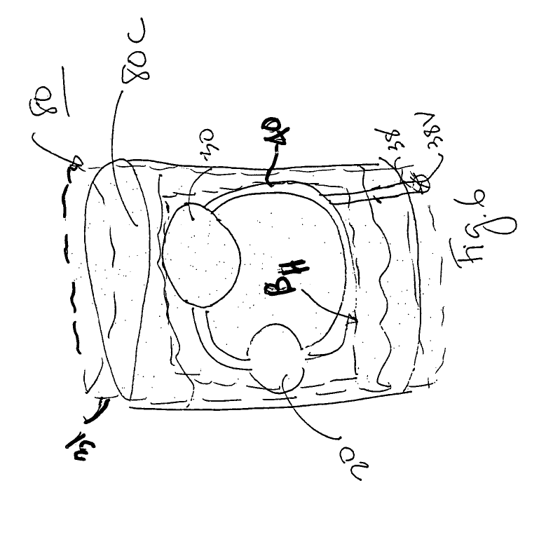

FIG. 5 is a block diagram of exemplary embodiments of data processing systems that illustrates systems, methods, and computer program products in accordance with embodiments of the present invention. The processor 310 communicates with the memory 314 via an address/data bus 348. The processor 310 can be any commercially available or custom microprocessor. The memory 314 is representative of the overall hierarchy of memory devices containing the software and data used to implement the functionality of the data processing system 305. The memory 314 can include, but is not limited to, the following types of devices: cache, ROM, PROM, EPROM, EEPROM, flash memory, SRAM, and DRAM.

As shown in FIG. 5, the memory 314 may include several categories of software and data used in the data processing system 305: the operating system 352; the application programs 354; the input/output (I/O) device drivers 358; a background estimator module 350; and the data 356. The data 356 may include image data 362 which may be obtained from an image acquisition system 320. As will be appreciated by those of skill in the art, the operating system 352 may be any operating system suitable for use with a data processing system, such as OS/2, AIX or OS/390 from International Business Machines Corporation, Armonk, N.Y., WindowsXP, WindowsCE, WindowsNT, Windows95, Windows98 or Windows2000 from Microsoft Corporation, Redmond, Wash., PalmOS from Palm, Inc., MacOS from Apple Computer, UNIX, FreeBSD, or Linux, proprietary operating systems or dedicated operating systems, for example, for embedded data processing systems.

The I/0 device drivers 358 typically include software routines accessed through the operating system 352 by the application programs 354 to communicate with devices such as I/O data port(s), data storage 356 and certain memory 314 components and/or the image acquisition system 320. The application programs 354 are illustrative of the programs that implement the various features of the data processing system 305 and preferably include at least one application that supports operations according to embodiments of the present invention. Finally, the data 356 represents the static and dynamic data used by the application programs 354, the operating system 352, the I/0 device drivers 358, and other software programs that may reside in the memory 314.

While the present invention is illustrated, for example, with reference to the Control Module for Convection Discharge and Recirculating Polarized Gas Hyperpolarizers 350 being an application program in FIG. 5, as will be appreciated by those of skill in the art, other configurations may also be utilized while still benefiting from the teachings of the present invention. For example, the Module 350 may also be incorporated into the operating system 352, the I/O device drivers 358 or other such logical division of the data processing system 305. Thus, the present invention should not be construed as limited to the configuration of FIG. 5, which is intended to encompass any configuration capable of carrying out the operations described herein.

In certain embodiments, the Control Module for Convection Discharge and Recirculating (Closed loop flow path) Polarized Gas Hyperpolarizers 350 includes computer program code for tracking polarization level data in the optical pumping cell and/or the reservoir and noting when the reservoir holds polarized gas that has decayed sufficiently that it is ready to be repolarized or is not suitable for use. The dispense can be prohibited if the polarization level is unduly low or an alternate dispense port may be activated (i.e., one upstream of the reservoir where fresher polarized gas can reside). The Module 350 can direct initiation of operations that will automatically determine and/or initiate controller operations that will do one or more of the following: (a) adjust the temperature in the pumping cell; (b) release polarized gas from the reservoir to a dispensing port; (c) adjust the flow rate of the gas in the closed loop path by adjusting the thermal gradient in the flow path or the valve positions of one or more flow valves; and (d) initiate replenishment or recharging of the closed loop path.

The I/O data port can be used to transfer information between the data processing system 305 and the NMR polarimetry system 320 or another computer system, a network (e.g., the Internet) or other device controlled by the processor. These components may be conventional components such as those used in many conventional data processing systems, which may be configured in accordance with the present invention to operate as described herein.

While the present invention is illustrated, for example, with reference to particular divisions of programs, functions and memories, the present invention should not be construed as limited to such logical divisions. Thus, the present invention should not be construed as limited to the configuration of FIG. 5 but is intended to encompass any configuration capable of carrying out the operations described herein.

The flowcharts and block diagrams of certain of the figures herein illustrate the architecture, functionality, and operation of possible implementations of probe cell estimation means according to the present invention. In this regard, each block in the flow charts or block diagrams represents a module, segment, or portion of code, which comprises one or more executable instructions for implementing the specified logical function(s). Certain of the flowcharts and block diagrams illustrate methods to operate hyperpolarizers or components thereof to yield polarized gas according to embodiments of the present invention. In this regard, each block in the flow charts or block diagrams represents a module, segment, or portion of code, which comprises one or more executable instructions for implementing the specified logical function(s). It should also be noted that in some alternative implementations, the functions noted in the blocks may occur out of the order noted in the figures. For example, two blocks shown in succession may in fact be executed substantially concurrently or the blocks may sometimes be executed in the reverse order, depending upon the functionality involved.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. In the claims, means-plus-function clauses, where used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. A method for producing hyperpolarized gas, comprising:
heating a target gas in an optical pumping cell, the optical pumping cell having opposing top and bottom portions;
polarizing the target gas in the optical pumping cell;
directing heated polarized gas to flow out of the top portion of the optical pumping cell to a storage reservoir, the storage reservoir having a temperature that is less than the temperature of the heated target gas flowing out of the optical pumping chamber; and
directing previously polarized gas from the reservoir into the optical pumping cell.

2. A method according to claim 1, wherein the second directing step is carried out by directing the previously polarized gas into a bottom portion of the optical pumping cell.

3. A method according to claim 1, wherein the reservoir has a greater volume than the optical pumping cell.

4. A method according to claim 1, wherein the optical pumping cell is heated to above about 150 degrees Celsius.

5. A method according to claim 4, wherein the reservoir is at room temperature.

6. A method according to claim 1, wherein the target gas is ³He.

7. A method according to claim 1, further comprising dispensing polarized gas from the reservoir.

8. A method according to claim 7, wherein the dispensing step is carried out during the polarizing step.

9. A method according to claim 1, further comprising providing a closed loop circulation path that extends between the optical pumping cell and the reservoir.

10. A method according to claim 9, wherein the circulation path, the reservoir and the optical pumping cell are held at a pressure of about 8-10 atm.

11. A method according to claim 1, wherein the second directing step has an associated flow rate of about 50 cc's/min.

12. A method according to claim 11, wherein the first directing step has an associated flow rate of about 50 cc's/min.

13. A method according to claim 1, wherein the optical pumping cell resides above the reservoir.

14. A method according to claim 1, wherein the optical pumping cell resides below the reservoir.

15. A method according to claim 1, wherein the optical pumping cell has a volume of about 250 cc's, and the reservoir has a volume of between about 250-1000 cc's.

16. A method according to claim 9, wherein the closed loop gas flow path has an associated total volume of between about 500-1250 including the volumes of the cell and reservoir, and wherein the volume of the closed loop gas flow path excluding the cell and reservoir volumes is about 15 cc's.

17. A method according to claim 1, wherein the first directing step is carried out using convective heating of the polarized target gas.

18. A method according to claim 10, further comprising generating a magnetic holding field that covers the optical pumping cell, the closed loop flow path and the reservoir.

19. A method according to claim 1, wherein the optical pumping cell is spaced apart from the reservoir by at least about 2-3 inches.

20. A method according to claim 1, further comprising determining the polarization level of the polarized gas held in the reservoir.

21. A method according to claim 20, further comprising determining the polarization level of the polarized gas in the optical pumping cell.

22. A polarizing system for polarizing a target gas via spin-exchange with an optically pumped alkali metal, comprising:
an optical pumping cell having separate exit and inlet ports, the exit port residing on a top portion of the optical pumping cell;
a reservoir chamber in fluid communication with the optical pumping cell, the reservoir having separate exit and inlet ports; and
a circulation gas flow path extending from the optical pumping cell exit port to the reservoir inlet port and the reservoir exit port to the optical pumping cell inlet port, wherein, in operation, target gas is polarized in the optical pumping cell and the polarized target gas convectively flows out of the optical pumping cell inlet port into the gas flow path to the reservoir chamber.

23. A system according to claim 22, further comprising a quantity of alkali metal held in the optical pumping cell.

24. A system according to claim 22, further comprising a quantity of target noble gas held in the optical pumping cell for polarization.

25. A system according to claim 22, wherein, during operation, the circulation gas flow path, the optical pumping cell, and the reservoir are all held at substantially the same operating pressure.

26. A system according to claim 25, wherein the pressure is between about 8-10 atm.

27. A system according to claim 22, wherein the reservoir is disposed below the optical pumping cell.

28. A system according to claim 22, wherein the reservoir is disposed above the optical pumping cell.

29. A system according to claim 22, wherein the optical pumping cell inlet port is positioned about a bottom portion of the optical pumping cell.

30. A system according to claim 22, wherein the reservoir has a larger volume than the optical pumping cell.

31. A system according to claim 22, further comprising a thermal source configured to heat the optical pumping cell to above about 150 degrees Celsius.

32. A system according to claim 22, wherein, during operation, the reservoir is at substantially room temperature.

33. A system according to claim 22, wherein the polarized target gas is polarized ³He.

34. A system according to claim 22, further comprising a dispensing port for dispensing polarized gas from the reservoir or from a location in the gas flow path downstream of the reservoir in advance of the optical pumping cell.

35. A system according to claim 22, wherein the gas flow path is configured to provide a polarized gas flow rate in the gas flow path of about 50 cc's/min.

36. A system according to claim 22, wherein the optical pumping cell and the reservoir define a thermal differential that is selected to provide a desired flow rate in the gas flow path.

37. A system according to claim 22, wherein, in operation, the polarized target gas travels through the gas flow path at a flow rate of about 50 cc's/min.

38. A system according to claim 22, wherein the optical pumping cell has a volume of about 250 cc's, and the reservoir has a volume of between about 250-1000cc's.

39. A system according to claim 22, wherein the circulation gas flow path has an associated total volume of between about 500-1250 cc's inclusive of the volume of the reservoir and cell, and wherein the circulation gas flow path has a volume of about 15cc's excluding the volume of the reservoir and cell.

40. A system according to claim 22, further comprising a magnetic field source for generating a magnetic holding field that covers the optical pumping cell, the gas flow path and the reservoir.

41. A system according to claim 40, wherein the optical pumping cell is spaced apart from the reservoir by at least about 2-3 inches.

42. A system according to claim 22, further comprising a RF surface coil held on the reservoir for determining the polarization level of the polarized gas held in the reservoir.

43. A system according to claim 22, further comprising a RF surface coil held on the optical pumping cell for determining the polarization level of the polarized gas in the optical pumping cell.

44. An apparatus for polarizing a target gas, comprising:
means for heating a target gas in an optical pumping cell, the optical pumping cell having opposing top and bottom portions;
means for polarizing the target gas in the optical pumping cell;
means for convectively directing heated polarized gas out of the top portion of the optical pumping cell to a storage reservoir, the storage reservoir having a temperature that is less than the temperature of the heated target gas flowing out of the optical pumping chamber; and
means for directing previously polarized gas from the reservoir into the optical pumping cell.

45. A computer program product for operating a hyperpolarizer with a laser excitation source, the hyperpolarizer employing convection-induced flow discharge of polarized gas from at least one optical pumping cell to at least one reservoir using a circulating gas flow path that includes a dispensing valve and a flow rate valve in fluid communication with the circulating gas flow path to produce polarized noble gas, the computer program product comprising:
a computer readable storage medium having computer readable program code embodied in said medium, said computer-readable program code comprising:
computer readable program code that determines the polarization level of polarized gas held in the optical pumping cell of the hyperpolarizer;
computer readable program code that determines the polarization level of polarized gas held in the reservoir of the hyperpolarizer; and
computer readable program code that in operation directs the hyperpolarizer to optically pump target gas and previously polarized recirculated target gas in the optical pumping cell.

46. A computer program product according to claim 45, wherein the computer program is configured to polarize ³He.

47. A computer program product according to claim 45, further comprising:
computer readable program code that monitors the temperature differential in the gas flow path between the optical pumping cell and a location proximate the reservoir; and
computer readable program code that can alter the temperature differential in the gas flow path to adjust the flow rate of the polarized gas in the circulation gas flow path.

48. A computer program product according to claim 45, further comprising computer readable program code that controls the operation of the at least one flow rate valve to automatically adjust the flow rate of the gas traveling through the gas flow path.

49. A computer program product according to claim 45, further comprising computer readable program code that activates a dispensing valve to dispense polarized gas to a user if the polarization level of the gas in the reservoir is determined to meet a desired level.
